(19) United States
(12) Patent Application Publication
(10) Pub. No.: US 2024/0331816 A1
(43) Pub. Date: Oct. 3, 2024

(54) CODE GENERATOR FOR ACCESSING DIFFERING HEALTH RECORD SYSTEMS

(71) Applicant: Cerner Innovation, Inc., Kansas City, MO (US)

(72) Inventors: Natalee Agassi, Blue Bell, PA (US); Raghavendra Ijjada, Overland Park, KS (US); Subhadeep Chakraborty, Kolkata (IN); Sourav Gantait, Kolkata (IN)

(73) Assignee: Cerner Innovation, Inc., Kansas City, MO (US)

(21) Appl. No.: 18/193,389

(22) Filed: Mar. 30, 2023

(51) Int. Cl. G16H 10/60; G06F 9/54

(52) U.S. Cl. CPC G16H 10/60; G06F 9/54

(57) ABSTRACT

Techniques for enabling a user, having little or no code-writing experience, to build and configure a user-customized software application configured to extract, process, and display information associated with user-selected health records or portions of health records is disclosed. The techniques may include operations such as receiving a selection of a first resource defined by a first EHR system, analyzing metadata associated with the first resource to identify a first set of characteristics corresponding to the first resource, identifying configuration fields associated with the first resource based on the first set of characteristics, presenting the configuration fields associated with the first resource, receiving configuration values for the configuration fields, and generating code for executing API calls to extract data associated with the first resource in accordance with the configuration values and for presenting the data associated with the first resource.

### TECHNICAL FIELD

The present disclosure relates to computer-implemented techniques for accessing information stored in health records. In particular, the present disclosure relates to an application built and customized to extract, process, and display information associated with user-selected health records.

### BACKGROUND

Applications typically include large amounts of code executable in association with computer programs or routines and configured to perform sundry actions and operations. As is well known, an application may provide access and management operations to a database or may generate and play media. The application may operate as a stand-alone function, or work in conjunction with additional applications. The application may execute on hardware, such as desktop computers, tablets, and phones, via virtual processors and/or distributed processors.

An application once initiated may display or cause display of a user interface, such as a Graphical User Interface (GUI), either locally or remotely, for presenting information to a local or remote user and may receive inputs from the user via the GUI based on interactions by the user with the presented information. The input from the user may indicate operations to be performed such as storage of information into or retrieval of information from a database. The database may contain Electronic Health Records (EHRs) stored at a health care provider facility by an EHR system of a health care provider facility. A health care provider facility may include, for example, a clinic or a hospital. However, the types of access, e.g., types of information, and options for processing information in the database are typically limited, or less than optimal, relative to the user's needs.

### BRIEF DESCRIPTION OF THE DRAWINGS

The embodiments of the disclosure are illustrated by way of example and not by way of limitation in the figures of the accompanying drawings. It should be noted that references to “an” or “one” embodiment of the disclosure in this disclosure are not necessarily to the same embodiment of the disclosure, and they mean at least one. In the drawings:

FIGS. 1A and 1B illustrate an operating environment in accordance with one or more embodiments of the disclosure;

FIG. 2 illustrates an example set of operations for enabling a user to build a custom application to access user-selected information associated with records of one or more EHR systems in accordance with one or more embodiments of the disclosure;

FIGS. 3A-3C depict example user interfaces for directing and receiving information, from a user device, relating to user preferences for building a custom application configured to access, process, and present data associated with one or more health care provider facilities or EHR systems;

FIG. 3D depicts an example display presented on a user device during execution of the custom application; and

FIG. 4 illustrates a block diagram that includes a computer system in accordance with one or more embodiments of the disclosure.

### DETAILED DESCRIPTION

In the following description, for the purposes of explanation, numerous specific details are set forth in order to provide a thorough understanding of the disclosure. One or more embodiments of the disclosure may be practiced without these specific details. Features described in one embodiment of the disclosure may be combined with features described in a different embodiment of the disclosure. In some examples, well-known structures and devices are described with reference to a block diagram form in order to avoid unnecessarily obscuring the present invention.
1. GENERAL OVERVIEW
2. ELECTRONIC HEALTH RECORD SYSTEMS
3. DATA EXTRACTION SYSTEM
4. APP BUILDER TO ACCESS DIFFERING HEALTH RECORD SYSTEMS
5. EXAMPLE EMBODIMENT
6. COMPUTER NETWORKS AND CLOUD NETWORKS
7. MICROSERVICE APPLICATIONS
8. HARDWARE OVERVIEW
9. MISCELLANEOUS; EXTENSIONS

### 1. GENERAL OVERVIEW

One or more embodiments generate code for extracting and presenting data associated with the resources defined by Electronic Health Record (EHR) systems. The system may present an interface with candidate data extraction targets and receive corresponding user input. As an example, the system accepts, as user input via the interface, a selection of an EHR system(s), and a selection of resources defined by the selected EHR system(s) for which data is to be extracted. The system may further accept user input including configuration values for configuring the extraction of resources and/or presenting extracted data corresponding to the resources. Based on the user input, the system generates and executes code that incorporates Application Programming Interface (API) calls with corresponding parameters to extract data from EHR system(s). A display of the extracted data may include information for user-selected resources/fields.

One or more embodiments customize and build a software application configured to extract, process, and display information associated with EHRs for users with little or no coding experience. The system customizes and builds the software application based on user input that defines the data to be extracted and/or defines attributes for the presentation of the extracted data.

One or more embodiments of the disclosure described in this Specification and/or recited in the claims may not be included in this General Overview section.

### 2. ELECTRONIC HEALTH RECORD SYSTEMS

Different health care provider facilities typically use and maintain different respective EHR systems. Data associated with an EHR system may include information within patient medical records of the EHR system. This data is typically stored in a non-standardized format with respect to data stored in other EHR systems associated with other health care provider facilities. Furthermore, each EHR system may be associated with a corresponding Application Programming Interface (API) that is different than APIs for other EHR systems. An application that is configured for extracting data based on a particular EHR system's data format and API may not be able to extract data from other EHR systems. Alternatively or additionally, the application may not be able to access sufficient information from the other EHR systems. Accordingly, different applications and/or different sets of code may need to be generated for accessing the data from different, respective EHR systems.

In view of these issues, health care providers continue to seek improved tools that include functionality to extract data from disparate sources such as different EHR systems. A combined dataset, from disparate sources, may be useful for healthcare providers to provide a continuity of care for individuals across multiple health care provider facilities. For example, the health care provider may wish to view in graphical form five blood-test parameters of an individual associated with the patient medical record. However, an application may enable viewing of a different number or type of blood-test parameters associated with the patient. Hence, the health care provider may be prevented from viewing data or information associated with the data corresponding to data considered most important by the health care provider for the health care provider's intended use of the data. Further, by virtue of different EHR systems inevitably using different standards or formats for their patient medical records data, interoperability typically is lacking regard to the application's ability to access differing patient medical records at divergent health care provider facilities.

### 3. DATA EXTRACTION SYSTEM

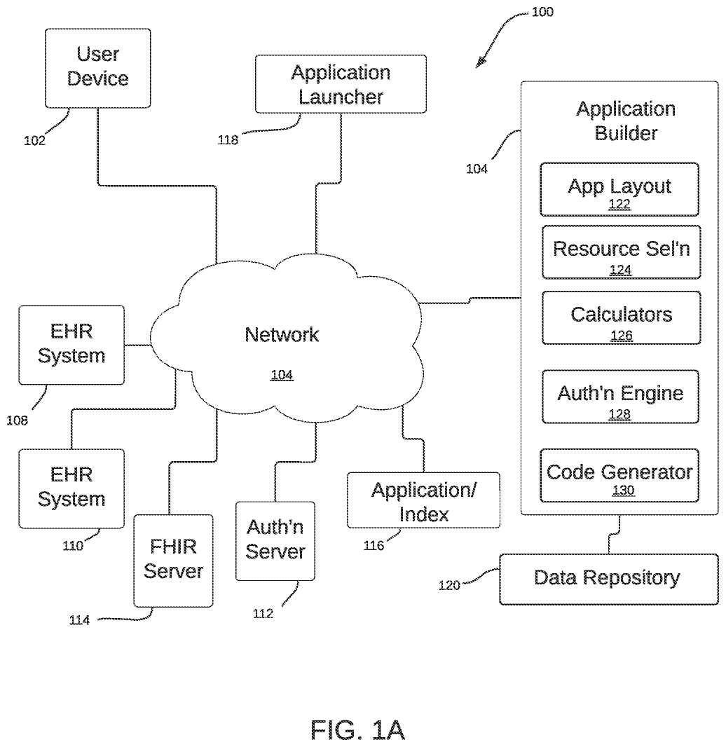

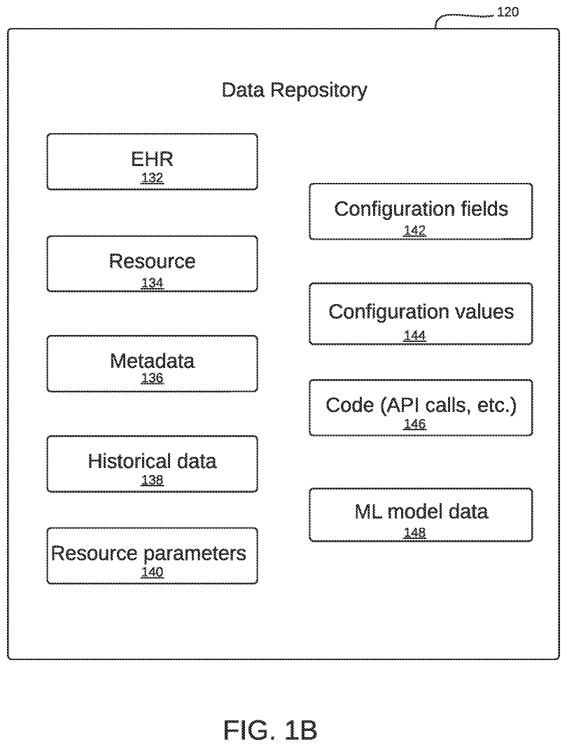

FIG. 1A illustrates an example of an operating environment suitable for practicing embodiments of the disclosure. An example computing system includes a combination of hardware and software elements for compiling and running an implementation of an application builder 106. The application builder 106 is executable for example on the computing system, the user device 102, or the EHR system 108, to create a custom application. The custom application, which may be executed for example on the computing system, the user device 102, or the EHR system 108, is enabled to access stored data according to individualized user-specified criteria selected during creation of the custom application. In the example embodiment of the disclosure shown in FIG. 1A, an operating environment 100 enables the building and execution of such a custom application by virtue of elements of the operating environment 100 including a user device 102 communicatively coupled via networking means (for example, network 104) to an application builder 106 and to one or more health care provider facilities or patient medical record databases such as EHR system 108 and EHR system 110.

The operating environment 100 as depicted includes a communicative coupling through network 104 between user device 102 that may implement a clinician user interface, and one or more of the EHR system 108 and the EHR system 110. It is further contemplated that some embodiments of the user device 102 may be directly communicatively coupled to, for example, the EHR system 108, the EHR system 110, or the application builder 106.

Embodiments of the user device 102 may take the form of a mobile communications device or a client computer including a user interface operated by a software application or set of applications. The user device 102 may include a physical device executing either or both of an application or a virtual machine. Examples of the user device 102 include a computer, a tablet, a laptop, a desktop, a netbook, a smart watch, a wearable smart device, a fitness tracker, a server, a web server, a network policy server, a proxy server, a generic machine, a function-specific hardware device, a hardware router, a hardware switch, a hardware firewall, a hardware network address translator (NAT), a hardware load balancer, and a mainframe. Further examples of the user device 102 may include a television, a content receiver, a set-top box, a printer, a mobile handset, a smartphone, a personal digital assistant (PDA), a wireless receiver and/or transmitter, a base station or access point, a communication management device, a controller, and/or a client device. In typical embodiments as described herein, the user device 102 includes a web-based application or set of applications that is usable to build a custom application and manage other user services provided by embodiments of the disclosure.

The operating environment 100 according to the present disclosure may have other arrangements not necessarily described herein or set forth in the examples yet recognizable as possible modifications or extensions by those skilled in the art. Other arrangements and elements (e.g., machines, user interfaces, functions, orders, and groupings of functions) can be used in addition to or instead of those shown or described, and some elements may be omitted altogether. Further, many of the elements described herein are functional entities that may be implemented as discrete or distributed components or modules or in conjunction with (e.g., within) other components or modules, and in any suitable combination and location. Various functions described herein as being performed by one or more components or modules may be carried out by hardware, firmware, and/or software. For instance, various functions may be carried out by a processor executing instructions stored in memory. Moreover, any number of components shown in FIG. 1A may be employed as part of or in communication with the operating environment 100 within the scope of the present disclosure. Each may be implemented via a single device or multiple devices cooperating in a distributed environment. Additionally, other components not shown may also be included within the operating environment 100.

The user device 102 in the illustrated embodiment of the disclosure is communicatively coupled via the network 104 to each of an authorization server 112, a Fast Health care Interoperability Resources (FHIR) device such as FHIR server 114, an application/index module 116 that may correspond to the custom application to be created, and an application launcher 118. Additionally, as elucidated in FIG. 1A, the application builder 106 may be communicatively coupled to or include a data repository 120, which is described below with reference to FIG. 1B.

The network 104 may include, without limitation, one or more local area networks (LANs) and/or wide area networks (WANs). Such networking environments are commonplace in clinics, hospitals, offices, enterprise-wide computer networks, intranets, and the Internet. In exemplary implementations of the disclosure, such networks comprise the Internet and/or cellular networks, amongst any of a variety of possible public and/or private networks. According to an embodiment, any one of the user device 102, application builder 106, EHR system 108, EHR system 110, authorization server 112, FHIR server 114, application/index module 116, and application launcher 118 may communicate with any other of the user device 102, application builder 106, EHR system 108, EHR system 110, authorization server 112, FHIR server 114, application/index module 116, application launcher 118 and/or data repository 120, e.g., via the application builder 106.

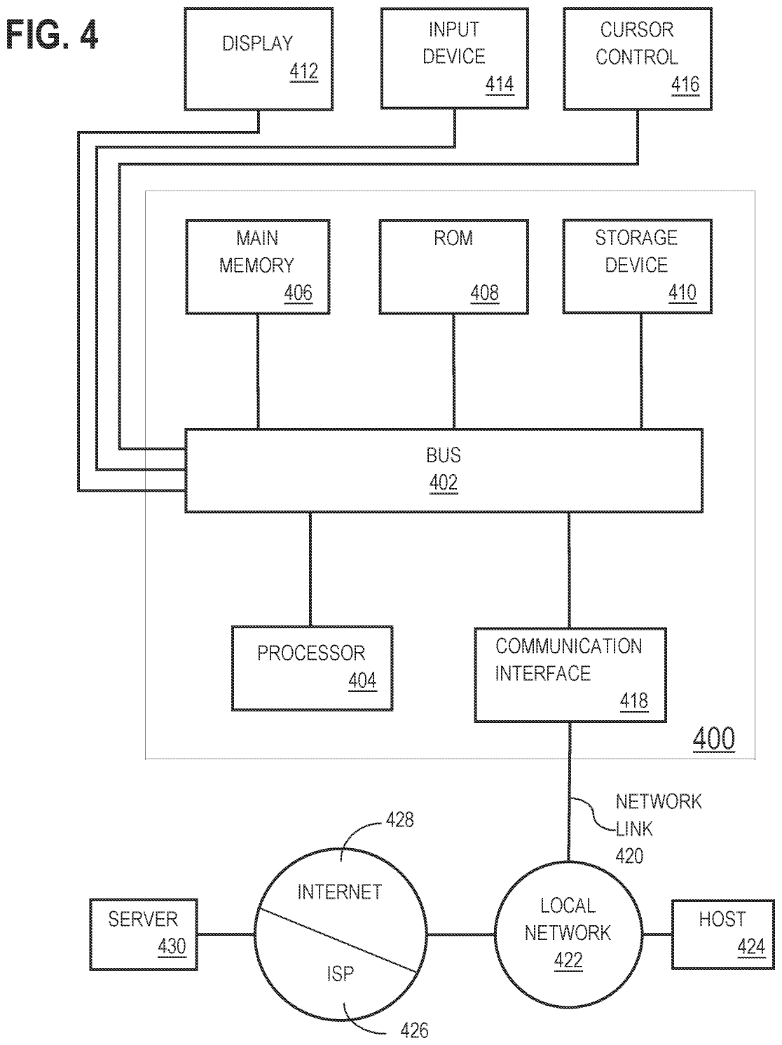

The application builder 106 may be included in or associated with a computing system, such as a computing system corresponding to computer system 400 (FIG. 4), which may be configured as a server communicatively coupled through network 104 to the user device 102, the EHR system 108, the EHR system 110, the authorization server 112, the FHIR server 114, the application/index module 116, the application launcher 118, and/or with the data repository 120 (e.g., via the application builder 106). The computing system comprises one or more processors operable to receive instructions and process them accordingly, and may be embodied as a single computing device or multiple computing devices communicatively coupled to each other. In one embodiment, processing actions performed by the computing system are distributed among multiple locations such as one or more local clients and one or more remote servers. In one embodiment, the computing system comprises one or more computing devices, such as a desktop computer, laptop, tablet, cloud-computing device, distributed computing architecture, ultra-mobile PC, or a mobile phone. In some embodiments, the computing system includes an adaptive multi-agent operating system, but it will be appreciated that the computing system may also take the form of an adaptive single agent system or a non-agent system. The computing system may include or operate in association with a distributed computing system, a distributed or centralized computing system, and/or a virtual computing system.

Embodiments of the computing system include a set of modules or components such as a computer software stack. The set of modules or components may facilitate creation of a custom application able to communicate with one or more of the EHR system 108 and the EHR system 110 according to standards or protocols known or promulgated in the industry. One or more of the authorization server 112, FHIR server 114, application/index module 116, application launcher 118, and/or data repository 120 may operate to enable creation via the application builder 106 of a custom application that is configured to operate or provide functionality compliant with the above described standards, protocols, and interoperability standards such as may be indicated or required with or by Substitutable Medical Applications, Reusable Technologies (SMART), FHIR, and/or SMART on FHIR. Further, the authorization server 112, the FHIR server 114, the application/index module 116, the application launcher 118, and/or the data repository 120 may operate in connection with, or may operate to build a custom application operable with or able to provide functionality that is compliant with, one or more of the authorization server 112, the FHIR server 114, the application/index module 116, and/or the application launcher 118. The application/index module 116 may be associated with or include an address or code associated with the custom application.

In the illustrated embodiment, the set, e.g., stack, is included in or under control of the application builder 106 as a component or module stack, e.g., operating as a distributed system on a virtualization layer of the computing system. The stack may include, without limitation, one or more of an application user-interface layout module 122, a resource/parameter selection module 124, a calculator-configuration module 126, an authorization engine 128, and a code generator 130. In some embodiments, one or more of the user-interface layout module 122, the resource/parameter selection module 124, the calculator-configuration module 126, the authorization engine 128, and the code generator 130 may comprise distinct components or may comprise components embodied as part of or within other components of the application builder 106 and/or of the operating environment 100. For example, one or more or a portion of one or more of the user-interface layout module 122, the resource/parameter selection module 124, the calculator-configuration module 126, the authorization engine 128, and the code generator 130 may be implemented in user device 102. Similarly, one or more parts or all of the user-interface layout module 122, the resource/parameter selection module 124, the calculator-configuration module 126, the authorization engine 128, or the code generator 130 may perform functions for two or more of the other components or modules referenced in the description of the operating environment 100.

Embodiments of the user-interface layout module 122, the resource/parameter selection module 124, the calculator-configuration module 126, the authorization engine 128, and the code generator 130 include one or more data stores of information described herein. Further, each of the user-interface layout module 122, the resource/parameter selection module 124, the calculator-configuration module 126, the authorization engine 128, and the code generator 130 may further include or be associated with one or more processors for performing various functions described herein. In some embodiments, the user-interface layout module 122, the resource/parameter selection module 124, the calculator-configuration module 126, the authorization engine 128, or the code generator 130 may be implemented as a cloud-based platform or may be distributed across multiple physical locations.

Execution of the application builder 106 may be initiated based on a signal, such as an input received from the user device 102 being operated by a health care provider. The application builder 106 develops/creates, with little to no code writing required or expended by the health care provider or user, a custom application. The custom application accesses the particular information sought after by the health care provider, information or data fields of a patient medical record at a health care provider facility or particular EHR system, such as EHR system 108. The custom application further accesses particular user-selected information or data fields of patient medical records at another health care provider facility or particular EHR system, such as EHR system 110.

The application builder 106 may receive inputs associated with accessing, configuring, and/or executing the application builder 106 via network 104 to create a custom application. The inputs may include signals from the user device 102 under control of a user at the user device 102 interacting with user interfaces presented via the application builder 106. The input(s) to the application builder 106 can be received during creation of the custom application. As described herein, the custom application once created is able to access stored data at one or more of a particular health care provider facility or a particular EHR system. The stored data may be accessed according to the above mentioned interoperability standards and according to user-identified resources and resource parameters specified based on input to the application builder 106. For example, SMART can standardize the process through which a custom application can access a particular data store and extract clinical information from a data store. The data store may be implemented by a health care provider facility and/or in a particular EHR system. As used herein, SMART on FHIR can define a workflow that the custom application can use to securely request access to data, and then receive and use that data. Further, SMART on FHIR as described and referenced herein can provide to the custom application standard, universal APIs for accessing electronic health information (EHI), e.g., at the particular data store.

The operating environment 100 may include any number of EHR systems such as EHR system 108 and EHR system 110. An EHR system, as referred to herein, may comprise or be associated with a hospital EHR system, an ambulatory clinic EHR system, a health plan EHR system, or a home monitor/patient monitor EHR system communicatively coupled to the network 104. In embodiments of the disclosure, an EHR system can include a health information exchange (HIE), a patient portal, a government database, a pharmacy database, or any other system capable of storing, receiving, transmitting, or the like, records or any health-related data.

The content available within an EHR system can include records of treatment events, medication history, diagnoses, problems, allergies, demographic attributes, summary of episode notes (SOEN), Clinical Document Architecture (CDA) documents, laboratory tests or results, time and data information, images, clinical notes, appointment notes, emergency contact information, clinical documentation of any kind, and any other health-related data, or any combination thereof for individuals. Different EHR systems can be disparate sources or, in other words, may be associated with different entities. For example, EHR system 108 may be associated with a hospital in Pennsylvania while the EHR system 110 may be associated with a pharmacy in Florida unrelated to source EHR system 110. Different EHR systems may utilize different standards or formats, e.g., Javascript Object Notation (JSON), Extensible Markup Language (XML), YAML Ain't Markup Language (YAML), Health Level 7 (HL7), and Consolidated Clinical Document Architecture (CCDA) by virtue of the EHR systems being disparate sources contained within and operated by the different entities. Furthermore, while the EHR systems are described herein as single sources (e.g., single databases), any of the EHR systems can each comprise multiple data stores associated respectively with one or many different entities. One of skill in the art will understand the EHR systems can take a variety of forms, be represented as multiple components, and communicate with any number of other sources.

Embodiments of the EHR systems may include data stores of health records, computers or servers that facilitate the storing and retrieval of the health records, and firewalls, e.g., a separate firewall associated with each EHR system. Furthermore, in some embodiments of the disclosure, an EHR system(s) may be implemented as a cloud-based platform or may be distributed across multiple physical locations. In some embodiments of the disclosure, an EHR system(s) includes record systems which store real-time or near real-time patient information, such as information associated with wearable, bedside, or in-home patient monitors.

It is further contemplated that embodiments of EHR systems use distinct clinical ontologies, nomenclatures, vocabularies, or encoding schemes for clinical information, or clinical terms. For example, EHRs of EHR system 108 may be affiliated with one hospital system that uses a nomenclature. Meanwhile, EHRs of EHR system 110 may be affiliated with another hospital system that uses a different nomenclature. Similarly, EHR system 108, which may be local with respect to a caregiver, patient, or clinician at the user device 102, may use one clinical nomenclature while EHR system 110, which may be remotely located with respect to the caregiver, patient, or clinician at user device 102, may use a differing nomenclature. Further, in some embodiments EHR system 108 and EHR system 110 are affiliated with two or more separate health care entities that use two or more distinct nomenclatures. Although FIG. 1A depicts multiple example EHR systems, it is contemplated that some embodiments may employ only one EHR system, or alternatively, may rely on a clinician user interface for storing or retrieving patient record information at a single data store, at the data repository 120, and/or at distributed locations.

Turning to FIG. 1B, the data repository 120 according to one or more embodiments of the disclosure comprises any type of storage unit and/or device (e.g., a file system, database, collection of tables, or any other storage mechanism) for storing data relevant to a clinic, hospital, or health care provider associated with the clinic or hospital, e.g., patient data. Further, the data repository 120 may include multiple different storage units and/or devices; the multiple different storage units and/or devices may or may not be of the same type or located at the same physical site. Further, the data repository 120 may be implemented by, or may execute on, the computing system (e.g., that includes the application builder 106). Alternatively, or additionally, the data repository 120 may be implemented or executed on a system separate from the application builder 106. In certain embodiments, the data repository 120 is populated with or accesses information from a variety of sources and/or systems.

As shown in FIG. 1B, the data repository 120 may store data or be able to access data such as: EHR data 132, resource data 134, metadata 136, historical data 138, resource parameters data 140, configuration fields data 142, configuration values data 144, code data 146, e.g., code associated with API calls or building a custom application, and machine learning (ML) model data 148. As used herein, EHR data 132 may refer to a collection of systematized patient or population electronically stored health information in digital format. EHR data 132 may include data associated with EHR systems or data used, e.g., created, written, or accessed, in connection with EHR systems; EHR data includes EHRs. As used herein, resource data 134 may include categories of or describing healthcare data. Resource data may include broad terms within or describing healthcare data including the following: patients, lab results, and observations. Resource data 134 can include data used to define or describe data elements, constraints on the data elements, and relationships among the data elements that may together make up an exchangeable electronic patient health record. As used herein, metadata 136 may include data that provides information about other data, such as data that describes and gives information about the other data. Metadata 136 can be used to provide structured types of references for the other data that enable sorting and identifying attributes associated with the other data.

As used herein, historical data 138 may include data collected about past events or circumstances pertaining to a particular subject, e.g., an event or resource. For example, historical data may include data collected in relation to prior execution(s) of the application builder 106. As used herein, resource parameters data 140 may include data relating to, e.g., describing, details, types, and uses of resources. For example, in accordance with the HL7 standard, the resource parameters for a “patient” resource may include “profiles” and “extensions.” In another example, resource parameters for the “patient” resource may include a read-access permission for that resource at a selected EHR system and/or a write-access permission for that resource at the selected EHR system. As used herein, configuration fields data 142 and configuration values data 144 may relate to user interfaces for enabling user-selection and user-configuration of user-selected resource parameters, e.g., for use in a custom application. For example, configuration fields data 142 and configuration values data 144 can be used for enabling user-selection and user-configuration inputs to define one or more user-selected resources as described below in connection with Operations 210-212 and with user interfaces 310-330. As used herein, code data 146 may include information used for creating or developing code as described herein, and may include the code itself.

Information stored in or by the data repository 120 may be structured. The structured data may include, for example, spreadsheets. The information may be unstructured. The unstructured data may include, for example, conversational text or social media posts. Further, the data repository 120 may store or access information including, included in, or related to variables associated with patient conditions or recommendations, recommendation or other knowledge bases, and recommendation rules or other rules. Moreover, the data repository 120 may store or access information including, included in, or related to recommendations data, recommendation update statistics, operational data, association rule bases, agent libraries, solvers, solver libraries, computer-usable instructions, patient-derived data, and health care provider information. Examples of operational data residing at, accessible by, or writable to the data repository 120 include events, frequent item sets (such as, for example, “X often happens with Y”), and item sets index information. It is contemplated that the data repository 120 may store any information that can be stored in a computer-storage device or system, such as user-derived data, computer usable instructions, software applications, or other information. Information stored at the data repository 120 may be implemented across any of components, modules, or elements within the operating environment 100. In the illustrated embodiment, for purposes of clarity and explanation this information is shown and described as being stored or residing in association with the data repository 120.

The data repository 120 in typical embodiments cooperates with the application builder 106, for example, to enable creation by the application builder 106 of a custom application. For example, information including one or more of EHR data 132, resource data 134, metadata 136, historical data 138, resource parameters data 140, configuration fields data 142, configuration values data 144, or code data 146 may be written to or read from the data repository in connection with the mentioned cooperating. The data repository 120 may cooperate with one or more custom applications once created, for example, in connection with performance of operations by the one or more custom applications. For example, information including one or more of EHR data 132, resource data 134, metadata 136, resource parameters data 140, configuration values data 144, and code data 146 may be written to or read from the data repository in connection with cooperating with the custom application(s).

In some embodiments, the data repository 120 may cooperate (e.g., directly or via one or more of the application builder 106 or a custom application) with other components, for example, to enable creation of a custom application or in connection with performance of operations by a custom application. For instance, the other components may include any of the authorization server 112, the FHIR server 114, the application/index module 116, or the application launcher 118. As an example, information including one or more of metadata 136, historical data 138, resource parameters data 140, code data 146, and ML model data 148 may be written to or read from the data repository 120 in connection with cooperating with the other components. As used in this and the above paragraph, cooperation with or by the data depository 120 may include: retrieving data or enabling access to data, creating data or enabling creation of data, and writing data or enabling writing of data at or by the data depository 120. In certain embodiments, the data repository 120 is populated with information from a variety of sources and/or systems.

Example operating environment 100 may include a clinician interface, e.g., at the user device 102, configured to facilitate communication, via the user device 102 being operated by a user, with one or more of the EHR system 108, the EHR system 110, and the application builder 106. Hence, embodiments of user device 102 may take the form of a user interface, e.g., the clinician interface, operated by a software application or set of applications of a client computing device, e.g., the user device 102, such as a mobile communication device or smartphone computing device. In one embodiment of the disclosure, the software application includes PowerChart® software, manufactured by Cerner Corporation. In one embodiment of the disclosure, the software application comprises a web-based application or applet. The software application or applet may facilitate accessing and receiving information from a user or health care provide, e.g., via EHR system 108 or the EHR system 110, about a specific patient or set of patients for which processing of a set of data and/or display of the set of data is to be performed and accessing the application builder 106. In some embodiments of the disclosure, user device 102 facilitates receiving orders for patients from clinicians, e.g., based on or associated with the processing of the set of data. For example, the user device 102 may display results associated with a user indicating that a given patient is likely to suffer from or to be suffering from a particular condition.

In one or more embodiments of the disclosure, by way of review, the operating environment 100 may include more or fewer components than the components illustrated in FIG. 1A, local to or remote from each other, implemented in software and/or hardware. Each component may be distributed over multiple applications and/or machines, and multiple components may be combined into one application and/or machine. Operations described with respect to one component may instead be performed by another component. Additional embodiments of the disclosure and/or examples relating to computer networks are described below in Section 6, titled “Computer Networks and Cloud Networks.” The application builder 106 comprises or facilitates a stack for providing functions of the application builder 106, such functions including or associated with an application (app) developer, e.g., a SMART FHIR application developer. One or more of the user-interface layout module 122, the resource/parameter selection module 124, the calculator-configuration module 126, the authorization engine 128, and the code generator 130 may be implemented in a system within the operating environment 100 apart from the computing system.

### 3. APP BUILDER TO ACCESS DIFFERING HEALTH RECORD SYSTEMS

Referring to the above described elements in FIG. 1A, the application builder 106 can create and leverage custom applications, e.g., SMART FHIR applications, with little to no coding on the part of clinicians and non-technical hospital staff. According to a particular aspect of the disclosure, each custom application, built via unique/particular user inputs to the application builder 106, can reduce burnout by allowing health care providers to work with one or multiple EHR systems to have a contextualized, persistent clinical view and to reduce searching for pertinent information in diverse EHR systems of various health care provider facilities.

In order to create the custom application according to the illustrated embodiment of the disclosure, a signal, e.g., from a user device 102 or another component that includes, is included with, or accesses the application builder 106 and a display, initiates execution of the application builder 106, e.g., via the network 104. According to some embodiments, the application builder 106 can be downloaded, e.g., to the user device 102, for execution, or can be executed remotely by the user device 102 via the network 104. Alternatively, in certain embodiments a device that includes the application builder 106, e.g., and a display, may access the application builder 106 in a more direct fashion. In an embodiment, the application builder 106 is a web-based application or applet.

An embodiment of the application builder 106 may comprise a software application, a set of applications, software agents, programs, modules, components, applications, routines, functions, or computer-performed services and/or may be implemented using belief-desire-intention (BDI) software models. Embodiments of the application builder 106 may reside on the computing system described above and/or on one or more servers in the cloud or distributed among the cloud and a client computing device, such as a personal computer, laptop, smartphone, tablet, mobile computing device, front-end terminal in communication with back-end computing system, or other computing device(s).

In response to execution of the application builder 106, by the user device 102 based on a user input to the user device 102, the application builder 106 may identify a particular health care provider facility or a particular EHR system associated with a health care provider facility with which the custom application to be created can interact. The identification may be based on a default value or setting, a setting corresponding to prior use of the application builder 106, data associated with the user at user device 102, or information associated in some other way with the current execution of the application builder 106 by the user device 102. For instance, the application builder 106 may initiate display of a user interface, e.g., GUI, on the user device 102 and, for enabling user input, the user interface may present elements such as icons, e.g., buttons or thumbnail images, text, symbols, other items or images, figures, patterns, or drop-down menus. These elements may be configured to allow inputs, e.g., via the user device 102, into data fields or to select among data items, e.g., associated with or stored in a database such as a data store at an EHR system, to be analyzed or otherwise processed by the application builder 106 and/or the custom application to be created by the application builder 106.

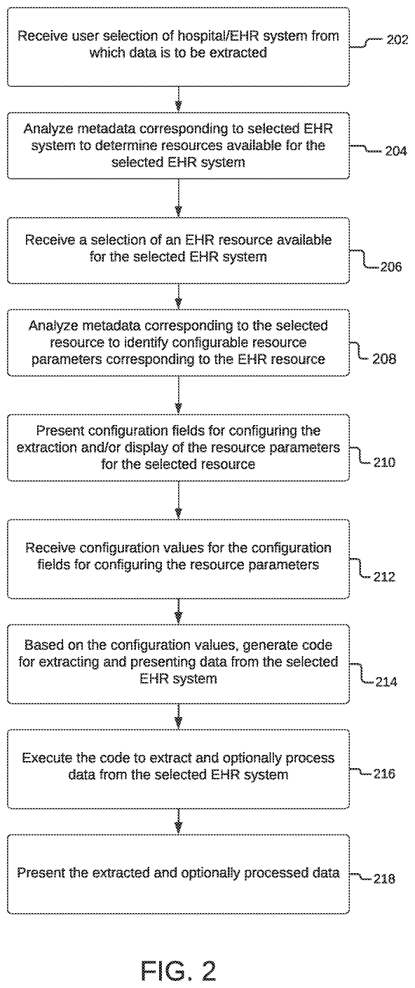

FIG. 2 illustrates an example set of operations performed by the application builder 106 to create a custom application with little to no code writing on the part of any user in accordance with one or more embodiments of the disclosure. In one or more embodiments of the disclosure, all or portions of one or more of the operations may correspond to, e.g., be performed by, hardware and/or software configured to perform the operations. One or more operations illustrated in FIG. 2 may be modified, rearranged, or omitted all together. Accordingly, the particular sequence of operations illustrated in FIG. 2 should not be construed as limiting the scope of one or more embodiments of the disclosure.

In one or more embodiments, the system, e.g., the application builder 106, receives user input selecting a particular health care provider facility or a particular EHR system from which data is to be extracted by the custom application to be created (Operation 202). In the illustrated embodiment of the disclosure, the application builder 106 initiates a display on the user device 102 inviting the user to enter a selection of a particular health care provider facility or EHR system to be associated with (e.g., linked, recognized, or otherwise in communication with) the custom application to be created. In certain embodiments of the disclosure, the application builder 106 may receive each of a user selection of a health care provider facility and/or EHR system from (to) which data is to be extracted (or modified or added) by the custom application.

An application enabling access by a device to information stored in the EHR system 108 may not be capable of enabling access by the device to information stored in the EHR system 110. The inability of the application to provide access to the EHR system 110 can be an unwanted consequence of formats or other properties differing among the EHR systems. The application builder 106 may receive each of a user selection of a health care provider facility and/or EHR system from (to) which data is to be extracted (or modified or added) by the custom application and may receive one or more additional user selections of one or more additional health care provider facilities and/or EHR system from (to) which data is to be extracted (modified or added) by the custom application.

The user interface may present a first menu of displayed elements prior to receiving user input selecting the particular health care provider facility or particular EHR system to be associated, e.g., for data extraction, with the custom application to be created. In response to the selection(s), e.g., via selection(s) from a drop-down list(s) associated with the first menu, the user interface may initiate display of an additional menu of additional displayed elements identified based on the selection(s) by a processor, e.g., at user device 102, executing the application builder 106. In the illustrated example, the additional menu is associated with information selected by the user via the first menu.

Hence, a user-input may be received by the application builder 106 to select multiple health care provider facilities or EHR systems to be accessible by the custom application (Operation 202). By way of review, data at the multiple health care provider facilities or EHR systems is typically stored in various formats rather than in a standardized format. Conventionally, there often is no easy way to pull, e.g., retrieve for processing, data from the multiple EHR systems because each EHR system varies in resources (i.e., data of patient medical records) of the EHR system, the format of those resources, etc. Embodiments of the disclosure recognize and leverage an expectation that all EHR systems will soon be required to implement/publish APIs that allow for extraction of data based on the FHIR standard promulgated by the standards development organization, Health Level Seven International, which also promulgated the HL7 standard. This means that data extracted from various EHR systems using their corresponding APIs will map to fields defined by the HL7 FHIR standard. For a given health care provider facility, e.g., hospital, the in-site EHR system may not necessarily include data for all the fields corresponding to a patient medical record, e.g., EHR, at the health care provider facility; however, the data that the patient medical records at the health care provider facility do have will be mappable to fields in the HL7 FHIR standard. The HL7 FHIR standard currently supports 145 resource types, typically in XML, JSON, or Terse Resource Description Framework (RDF) Triple Language formats.

Based on each selected health care provider facility or EHR system, the application builder 106 initiates a determination of resources available for use by the custom application to be created (Operation 204). The resources available for use by the custom application to be created typically will vary based on the particular health care provider facility or EHR system. In the illustrated example, the resources available for use may be identified by analyzing metadata, e.g., attributes, corresponding to the selected health care provider facility or EHR system, e.g., based on an FHIR standard. This metadata may describe particular resources available for use, by the custom application to be created, according to the HL7 FHIR standard. Here, each resource available at the selected health care provider facility or EHR system can correspond to a packet of information usable to exchange or store data that satisfies most use cases in the clinical setting for the selected health care provider facility or EHR system.

In the example embodiment of the disclosure, analyzing the metadata corresponding to the selected health care provider facility or EHR system identifies the resource “user” as an available resource. The resource “user,” or “practitioner,” may cover, e.g., pursuant to HL7, a person who is directly or indirectly involved in the provisioning of health care or health care-related services as part of his or her formal responsibilities. The resource is used for attribution of activities and responsibilities associated with individuals who may be engaged in pursuits including physician, dentist, pharmacist, physician assistant, nurse, scribe, midwife, dietitian, therapist, optometrist, paramedic, medical technician, laboratory scientist, prosthetic technician, radiographer, social worker, professional home carer, official volunteers, receptionist handling patient registration, or IT person merging or unmerging patient records. A practitioner or user may perform different roles within the same or different organization or health care provider facility. Depending on jurisdiction and custom, it may be necessary to maintain a specific resource for each such role or have a single resource, e.g., practitioner, with multiple roles. The resource is not used for persons without a formal responsibility, e.g., an individual taking care for a friend or relative; such individuals may be described by alternative resources such as a “related person” resource to the extent performing some action or being referenced by another resource. Concepts not included within the HL7 definition of a resource may be found in HL7 defined or referenced resource parameters such as “patient's contact.”

Analyzing metadata corresponding to the selected health care provider facility or EHR system may also identify the resource “patient” as available. The resource “patient” may cover, e.g., pursuant to HL7, demographic and administrative data about a patient at the health care provider facility receiving care. The resource describes a wide range of patient health-related activities including curative activities, psychiatric care, social services, pregnancy care, nursing and assisted living, dietary services, and tracking of personal health and exercise data. Data in this resource covers the “who” information about a patient: its attributes are focused on the demographic information necessary to support administrative, financial, and logistic procedures. Noting that a patient medical record is generally created and maintained by each organization providing care for a patient, a patient receiving care by multiple health care providers or at multiple health care provider facilities may have his or her information present in multiple patient resources. Concepts not included within the HL7 definition of a “patient” resource, such as in the current example race, ethnicity, organ donor status, nationality, may be found in associated resources or in HL7 defined resource parameters such as “profiles” or “extensions” used with or related to the “patient” resource.

Continuing with Operation 204, based on the resources determined by analyzing the metadata, the application builder 106 may present the determined resources to the user for review, alteration, or selection. For example, the application builder 106 may initiate display of the determined resources, via the user interface as part of or associated with a second menu, for user review, modification, or selection. For instance, the second menu may include a drop-down menu displaying, for selection by the user, elements corresponding to all or a portion of the resources determined to be available at the selected health care provider facility or EHR system.

In an embodiment, the application builder 106 receives one or more selections of the resources presented by the application builder 106 in the second menu of resources (that were determined based on analyzing the metadata corresponding to the selected health care provider facility or EHR system) (Operation 206). For example, the application builder 106 receives, via interaction by the user with the user interface, selections of resources that were presented by the application builder 106 in the second menu of resources.

Noting that the current HL7 FHIR standard supports 145 resource types, the listing in the second menu may display, for selection by the user, a subgroup of the total resources determined to be available. In an example embodiment of the disclosure, the resource(s) presented in the second menu correspond to subset(s) of the more general resources identified, e.g., based on metadata, from a total set of resources determined, by the application builder 106 at Operation 204, to be available at the selected health care provider facility or EHR system. The subset of resources may include one or more of practitioner (or user), patient, administrative, organization, location, coverage, and invoice. In an embodiment of the disclosure, Operation 206 includes receipt by the application builder 106, via interaction of the user with the user interface, of a selection from the subset of resources (presented by the application builder 106 in the second menu of resources).

Based on this selection by the user from the subset of resources, and/or on the selection(s) of a health care provider facility or EHR system, the application builder 106 may present to the user an additional menu containing additional resources, which were determined to be available for the selected health care provider facility or EHR system but which were not presented in the second menu. In an example where the subgroup of resources presented in the second menu contain the three resources of patient, laboratory results, and insurance claims, and where only the first resource of these three resources is selected, an additional menu may be presented. The additional menu may be based on the user selection of “patient,” containing the three resources of medications, workflow, financial, each relating to “patient” and selectable via input to the user interface (e.g., by input from the user device 102 being operated by a user). Hence, the application builder 106 may present resources determined to be available, in an iterative, sequential, or otherwise multi-menu fashion and/or the application builder 106 may receive selections to the presentations of resources in an iterative, sequential, or otherwise in a multi-menu response fashion.

One or more further menus may be presented by the application builder 106 using the user interface to present further resources, e.g., determined to be available for a custom application to be created, based on the menu selections made via the additional menu, the second menu, and/or the first menu and/or based on analyzing metadata associated with the menu selections. The further menu(s) may present: resources determined to be subcategories of, or more particular resources with respect to, one or more of the resources selected via the second menu or via the additional menu. For example, a further menu may present additional resources which were determined to be available for the selected health care provider facility or EHR system but which were not presented in the second menu or the additional menu.

In an example embodiment, resources corresponding to more generalized categories, such as one or more of patient, laboratory results, and insurance claims, determined among others by analyzing the metadata, are presented in the second menu, followed by presentation by the application builder 106 of additional resources, e.g., in an additional menu, where the additional resources are also determined by analyzing the metadata based on the selected health care provider facility or EHR system. Each of the resources in the additional menu may further, or alternatively be determined by analyzing metadata associated with one or more of the resources selected by the user via the second menu.

In example embodiments of the disclosure including additional menus or including further menus, based on a resource “patient” selected by the user via the second menu and optionally based on metadata associated with the “patient” resource, the additional menu may present for selection by the user, e.g., pursuant to HL7 standards, one or more resources from a group including: clinical, diagnostics, medications, workflow, or financial. Moreover, based on a resource “clinical” selected by the user via the second menu and optionally based on metadata associated with the “clinical” resource, the additional menu may present for selection by the user, e.g., pursuant to HL7 standards, one or more resources. These one or more resources may be selected from a group including: allergyintolerance, condition (problem), procedure, familymemberhistory, careplan, goal, careteam, clinicalimpression, adverseevent, detectedissue, and riskassessment. Further, based on a resource “diagnostics” selected by the user via the second menu and optionally based on metadata associated with the “diagnostics” resource, the additional menu may present for selection by the user, e.g., pursuant to HL7 standards, one or more resources from a group including: observation, report, specimen, imagingstudy, genomics, specimen, and imagingstudy.

Further, a resource “medications” selected by the user via the second menu may cause, e.g., based on the selection of the “medications” resource and optionally based on metadata associated with the “medications” resource, the application builder 106 to present an additional menu to the user containing, e.g., pursuant to HL7 standards, one or more resources from a group including: medication, request, dispense, administration, statement, and immunization. Also, based on a resource “workflow” selected by the user via the second menu and optionally based on metadata associated with the “workflow” resource, the additional menu may present for selection by the user, e.g., pursuant to HL7 standards, one or more resources from a group including: introduction, task, appointment, schedule, referral, and plandefinition. Additionally, based on a resource “financial” selected by the user via the second menu and optionally based on metadata associated with the “financial” resource, the additional menu may present for selection by the user, e.g., pursuant to HL7 standards, one or more resources from a group including: claim, account, invoice, chargeitem, coverage, eligibility, request, response, and explanationofbenefit.

At Operation 208, based on the selection of one or more resources received, e.g., at Operation 206, application builder 106 identifies configurable resource parameters corresponding to each of the received one or more selections of resources. For example, application builder 106 analyzes metadata corresponding to each resource selection received to identify a corresponding one or more configurable resource parameters for the selected resource. For instance, a third menu may include a drop-down menu displaying, for selection by the user, elements corresponding to configurable resource parameters available for the selected “patient” resource. The configurable resource parameter(s) for “patient,” for example, may include a selection of one or more of read-access to data at a selected health care provider facility or EHR system corresponding to the HL7 FHIR resource “patient” and/or write-access to data at the selected health care provider facility or EHR system corresponding to the HL7 FHIR resource “patient.

In an embodiment, the application builder 106 presents one or more configuration fields offering corresponding selections to the user for configuring the configurable resource parameters (Operation 210). The one or more configuration fields may enable selections for configuring operations that may be performed by or in association with execution of the custom application to be created. These operations may include one or more of the extraction, modification, or presentation of one or more of the selected resources. The operations may be associated with the extraction, modification, and/or presentation of data associated with: (a) the selected health care provider facility or EHR system, (b) the resources selected via any of the second menu, the additional menu, or the further menu, and/or (c) details or options with respect to implementing the configurable resource parameters identified in Operation 208. In an example embodiment of the disclosure, for each of the configurable resource parameters identified in Operation 208, the application builder 106 presents a third menu or menus relating to configuring operations that may be performed by the custom application on the selected resources or information associated with the selected resources. The third menu(s) may display via the user interface configuration options, e.g., configuration options in the form of one or more third menus, such as one or more drop-down menus and one or more input text boxes enabling input of information (e.g., via the user device 102 being operated by a user).

The application builder 106 receives one or more inputs, e.g., selections, of configuration values corresponding to the configurable resource parameters presented by the application builder 106 in the third menu of configurable resource parameters (Operation 212). The resource parameters including parameters that were determined based on the application builder 106 analyzing metadata corresponding to each resource selection received to identify the corresponding one or more configurable resource parameter(s) for the selected resource). For example, the application builder 106 receives, via interaction by the user with the third menu displayed via the user interface, inputs indicating, e.g., specifying, configuration values for the configurable resource parameters that were presented by the application builder 106 in the third menu of resources.

Based on the configuration values, the application builder 106 generates code for extracting, modifying, or presenting data that is associated with or from (i) the selected health care provider facility or facilities and/or (ii) the selected EHR system or systems (Operation 214). For example, code may be generated to enable a custom application to perform operations including the extraction, modification, or presentation of data associated with one or more selected health care provider facilities or EHR systems, with the resources selected via any of the second menu, the additional menu, or the further menu. Alternatively or additionally, code may be generated to enable a custom application to perform operations associated with the configuration values selected via the third menu and/or details or options with respect to implementing the configurable resource parameters and/or configuration values. All or a subset of the menus, e.g., of the first, second, additional, further, and third menus, or any portions thereof, may be presented in various combinations, at the same time or at differing times, and/or in differing arrangements, forms, or orders. In some embodiments, the application builder 106 sends to the user device 102 or to another device associated with creation of the new custom application, a link identifying, e.g., by virtue of a Uniform Resource Locator (URL), a web address of the newly created custom application.

As described above, the application builder 106 may provide functions including or associated with an application (app) developer, e.g., a SMART FHIR application developer, to create a custom application. Code generated, e.g., accessed, modified, and/or developed, by the application builder 106 may create or facilitate creation of one or more custom applications or part(s) of one or more custom applications. In embodiments, during creation of the custom application(s), the application builder 106 may automatically generate code corresponding to needed FHIR calls, e.g., based on the HL7 FHIR standard, to enable fetching/extraction of data during execution of the custom application. For instance, based on selections made via inputs, e.g., from the user device 102, the code generator 130 of the illustrated example generates needed API calls, e.g., FHIR based calls, to extract/retrieve and process user-selected data to be displayed during subsequent execution of a custom application. Hence, embodiments of the present example may harness mappings to FHIR concepts as indicated or needed.

Techniques and systems for generating, e.g., accessing, modifying, and/or developing, code that may facilitate creation of a custom application may be based on known technologies or applications, or modifications or extensions thereof, pertaining for example to code generation. For instance, techniques and systems for generating code (e.g., for performing the extracting, modifying, and presenting of EHR data as elucidated herein with reference to Operations 216-218) to create a custom application may correspond to those used or referenced in connection with the Oracle Application Express (APEX) platform provided by Oracle Corporation of Redwood Shores, California. Other techniques and systems for generating the code to create a custom application may correspond to concepts and techniques known in the art including, for example, those used or referenced in connection with U.S. Pat. No. 9,373,094 and the SMART on FHIR platform (smarthealthit.org) provided by the Computational Health Informatics Program, Boston Children's Hospital, Boston, Massachusetts, the contents of all which are incorporated herein. The code generated by the application developer 106 may configure a custom application to implement operations associated with, e.g., defining, certain clinical information representations of or relating to extracted data in a variety of ways, tables, summarizations, and sub-summarizations according to individual, e.g., unique, needs or objectives associated with user inputs received by the application builder 106 during creation of the custom application.

Code used by the application builder for building custom applications can be stored, e.g., at the data repository 120, in or in association with one or more libraries of reusable resources, components, source files, and/or GUI controls (such as Drag and Drop GUI builders). For instance, the source files that may be created, modified, stored, and retrieved may include files that contain (e.g., as a starting point for a system of processing) program instructions, source code, original data, or essential data. In an example, a set of code is predefined for each EHR system and the set of code is selected based on a selected EHR system, e.g., at Operation 202. The set of code may include API calls as described herein. One or more of the computing system, the user device 102, the EHR system 108, or another component may store code associated with resource selections as variables that are then used by a custom application once created as parameters for the API calls. Further, one or more of the computing system, the user device 102, the EHR system 108, or another component may generate code that incorporates authentication keys for enabling a custom application once created to access, for example, health care provider facilities or EHR systems.

In one or more embodiments, the system executes the code to extract the data from a selected EHR system (Operation 216). Additionally or alternatively, the system in certain embodiments executes the code to extract and process the data from a selected health care provider facility or EHR system. Executing the code may be initiated by a signal instructing opening (e.g., execution) of a custom application as described herein. The signal may be received at or from a health care provider facility (e.g., received from an EHR system of a health care provider) or from another location (e.g., received at or from one or more of the computing system, the user device 102, the EHR system 108, or another component). For example, the signal may be generated in response to a double-click action performed via a pointing device on an image, icon, button, or text associated with (e.g., representing) the custom application. Based on user-inputs received by the application builder 106 during creation of the custom application, processing by the custom application during or in association with execution of the generated code initiates operations such as API calls with corresponding parameters to, e.g., extract data from EHR system(s). The API calls may correspond to standards or protocols associated with, e.g., SMART FHIR applications, or other standards, or other protocols. See, e.g., SMART on FHIR platform (smarthealthit.org) referenced above.

In one or more embodiments, one or more of the computing system, the user device 102, the EHR system 108, or another component as described herein presents the extracted data and optionally processed data associated with the extracted data (Operation 218). Additionally or alternatively, one or more of the computing system, the user device 102, the EHR system 108, or another component in certain embodiments may present at least a portion of the extracted data or information indicative of the extracted data and further may present at least a portion of the processed data or information indicative of the processed data. One or more of the computing system, the user device 102, the EHR system 108, or another component may present one or both of the extracted data and the processed data in a default or a user-selected display format or style (e.g., based on one or more of prior use by a user or device, historical data 138, ML model data 148, and ML, AI model data, neural network data, and adaptive agents).

A presentation, e.g., display, operation may be performed under control of or in connection with a custom application being executed. The presentation operation may be initiated or performed at or by one or more of the computing system, the user device 102, the EHR system 108, or another component in certain embodiments and may present at least a portion of the extracted data or information indicating the extracted data. Alternatively or additionally, the presentation operation may present at least a portion of the processed data or information indicative of the processed data. For example, a presentation operation may display data retrieved from or otherwise associated with a selected health care provider facility or EHR system, e.g., data extracted from one or both of EHR system 108 and EHR system 110.

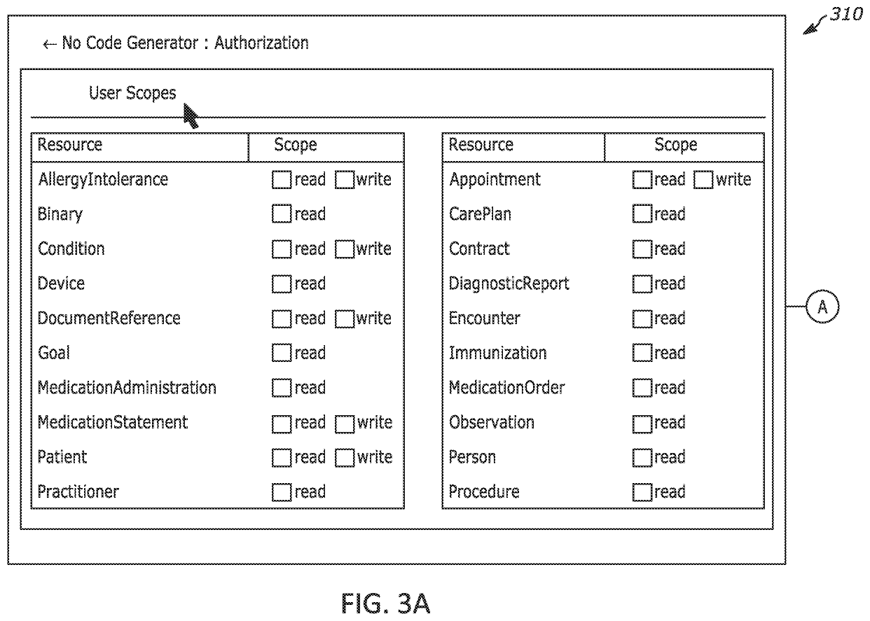

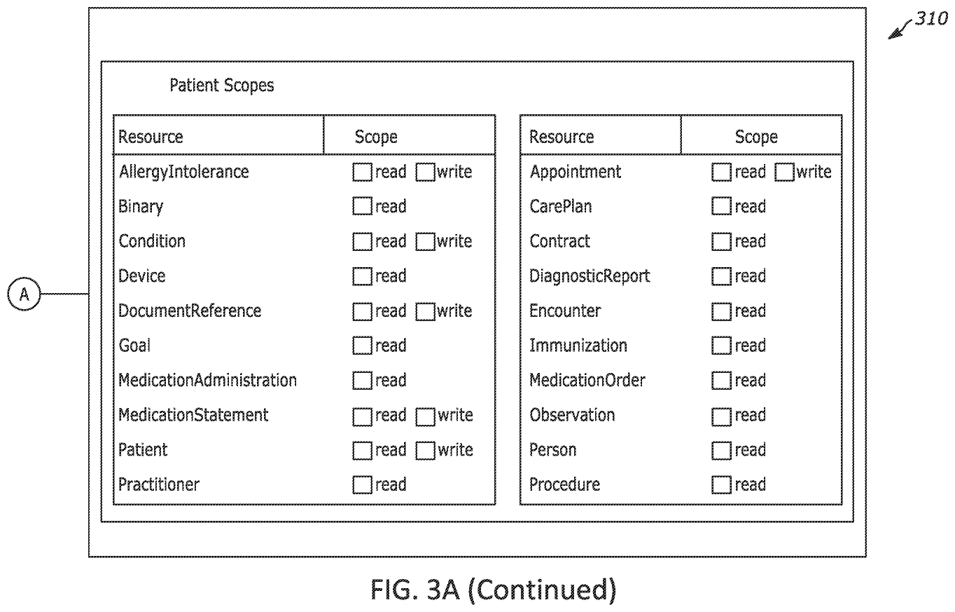

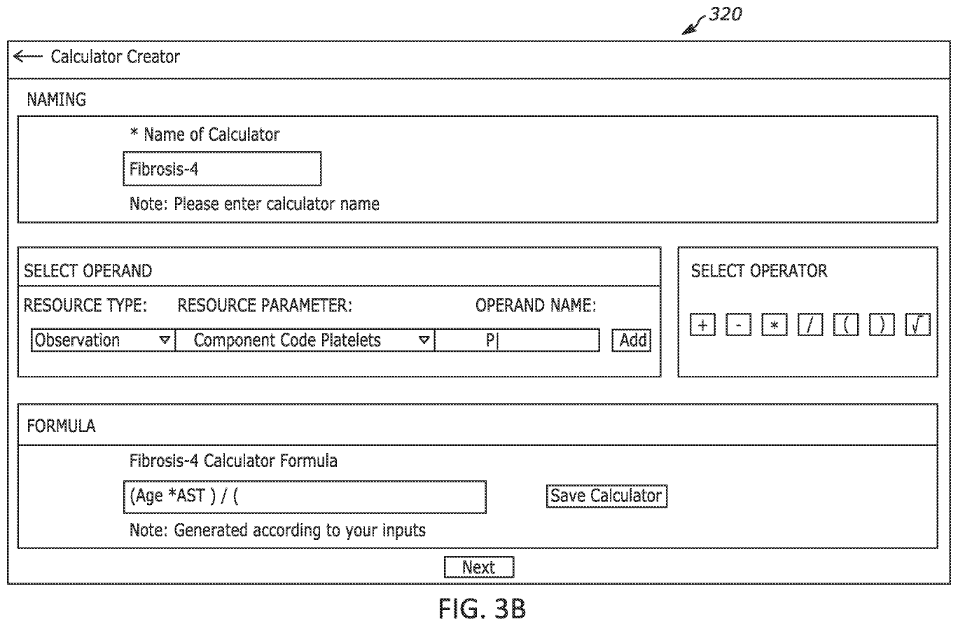

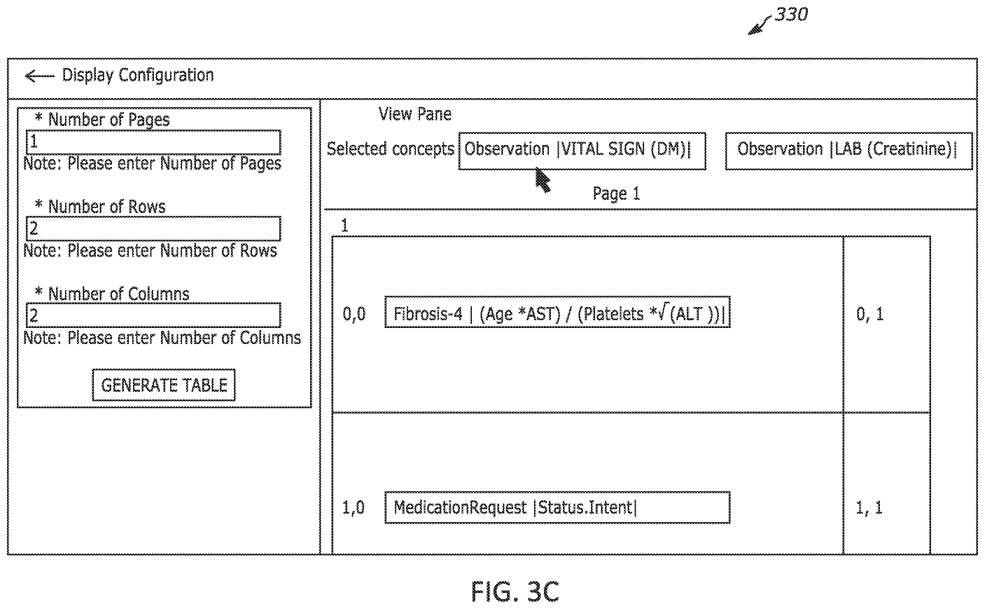

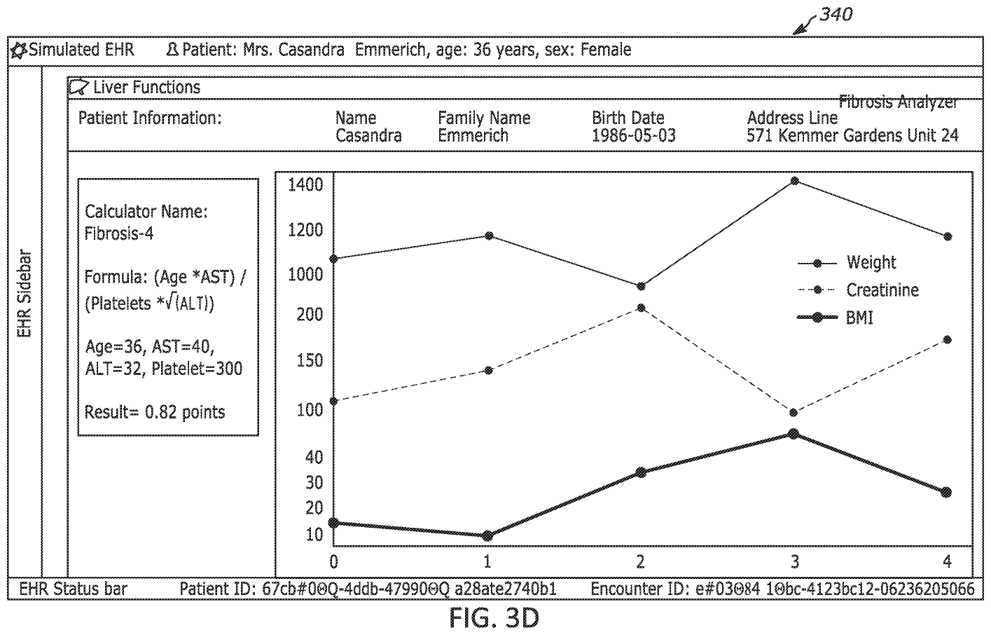

In an example embodiment, a presentation operation, e.g., corresponding to operation 218, may display content in accordance with user-selected parameters, such as a number of pages, a number of rows, and a number of columns, e.g., as shown and described in connection with the example display 340 of FIG. 3D. In the example display 340, user-input(s) to the application developer 106 have defined a field (0,0) to present, by a custom application during execution, data from a selected health care provider facility or EHR system extracted and processed according to a user-defined “Fibrosis-4” calculator, e.g., as defined via a user interface 330 of FIG. 3C.

The presentation operation in the example embodiment may further include user-defined fields (0,1), (1,0), and (1,1) configured to present data (from a selected health care provider facility or EHR system extracted and processed) according to, e.g., the user-defined additional calculators: “Observation|VITAL SIGN (BMI),” “MedicationRequest,” and “Observation|LAB (Creatnine),” respectively. In addition or alternatively, other calculators (e.g., routines), objects, or other processes defined (e.g., via the user device 102 being operated by a user) or obtained elsewhere may be presented during execution of a custom application. As an example, the other routines, objects, or processes may correspond to, e.g., include, any application available to a user or capable of being made available by a user for use during creation of the custom application. Such routines may include items or objects included in or associated with content of this disclosure such as the algorithms, applications, BDI software models, components, components embodied as part of or within other components, computer-performed services, elements, functional entities, functions, ML algorithms, modules, operations, processes, processes corresponding to hardware, firmware, and/or software, programs, routines, agents, and supervised or unsupervised components.

In one or more embodiments of the disclosure, an ML algorithm, e.g., based on or corresponding to the ML model data 148 at the data repository 120, can be included in or made accessible, e.g., via the user interface 330, to a particular custom application under development (being created) by the application builder 106. For example, the ML algorithm may be iterated to learn a target model f that best maps a set of input variables to an output variable, e.g., using a set of training data. For instance, such an ML algorithm in embodiments can be configured to generate and/or train, during execution of the custom application, an urgent care or emergency room model. The training data, e.g., stored with the ML model data 148 at the data repository 120 can include datasets and associated labels and code associated therewith. These datasets in turn can associated with input variables for the target model f, and the associated labels are associated with the output variable of the target model f during execution of the custom application.

The training data may be updated during execution of the custom application based on, for example, feedback on the accuracy of the current target model f. Updated training data, e.g., stored with the ML model data 148 at the data repository 120, may be fed back into the ML algorithm during execution of the custom application which in turn updates the target model f. The target model f can be applied to the datasets of the training data, during execution of the custom application, and a maximum number of results may be determined by the target model f as matches for the labels of the training data. Further, according to an aspect of the present disclosure, the generated code corresponding to a target model f, e.g., configured to provide a best fit of datasets of training data to labels of the training data, can be applied to datasets corresponding to the training data to identify desired datasets. Different target models may be generated based on different ML algorithms and/or different sets of training data.

As described herein, each ML algorithm may include supervised components and/or unsupervised components. Various types of algorithms may be used, such as linear regression, logistic regression, linear discriminant analysis, classification and regression trees, naïve Bayes, k-nearest neighbors, learning vector quantization, support vector machine, bagging and random forest, boosting, backpropagation, and/or clustering.

FIG. 3D illustrates an example display 340 presented to a user, e.g., on the user device 102, during execution of a custom application created by the application builder 106, e.g., based on (using) the user interface 310, the user interface 320, and the user interface 330. The display is based on the user-customized calculator, e.g., “Fibrosis-4,” defined via the user interface 320 and configured to enable processing, during execution of the custom application, using both user-selected extracted data and the user-defined formula corresponding to the “Fibrosis-4” calculator. For example, the display 340 includes a user-defined plot, e.g., defined via the user interface 320 and the user interface 330, elucidating a sample processing by the custom application of information, e.g., data extracted from one or both of EHR system 108 and EHR system 110, based on the user-defined “Fibrosis-4” calculator and based on the Operations 202-212. In the example embodiment, the “Fibrosis-4” calculator has been constructed, e.g., via the user interface 320, to effectuate the formula “(Age*AST)/(Platelets*V (ALT))” via code generated by the application builder 106, e.g., at Operation 214.

Some embodiments of these methods facilitate decision making by dynamically displaying, during execution of a custom application (e.g., created via inputs from the user device 102 under control by a user), user-relevant patient information. The user-relevant patient information may indicate, pertain to, be extracted from, or be derived based on one or more of (i) the EHR system 108, (ii) the EHR system 110, (iii) the data repository 120, (iv) other information, concepts or knowledge derived from (i)-(iii), or (v) other items such as information indicating a patient condition or a recommendation of a treatment for the patient. The above described electronic models, electronic adaptive agents, and/or electronic models including electronic adaptive agents may be used to determine, e.g., by virtue of implementing techniques known to those skilled in relevant arts including data processing, data synthesizing, and data learning arts, the user-relevant patient information. For example, a clinician interface generated at user device 102, e.g., during execution of a custom application, may facilitate accessing and receiving information about a specific patient or set of patients, receiving information such as patient information, selections, queries, commands, or actions, and displaying results, recommendations, or orders. In some embodiments, the clinician interface facilitates receiving orders for the patient from a clinician/user, based on the results. In some embodiments, the clinician interface comprises a GUI, e.g., under or associated with operation of a custom application as described herein, configured to facilitate clinical decision support and/or to provide diagnostic or update services such as creating, evaluating, or modifying condition programs, prediction models associated with health-condition programs, libraries, and/or content tables used for agents. The GUI may be configured for facilitating human confirmation of computer-derived determinations such as actions, recommendations, diagnoses, linkages, or other such services.

In embodiments of the disclosure, the EHR system 108 for example may be FHIR compliant as described above and configured to be mapped to one or more of patient medical records associated with a particular hospital or patient medical records associated with an HIE platform and related to immunization status. In some embodiments, retrieving by the custom application from the EHR system resources that correspond to patient data artifacts associated with at least one record of the patient in the EHR system may be performed via any one or more of the above described models or adaptive agents such as by applying ML and/or an AI model to the information. As described above. the operations described herein may comprise executing a machine-learning model, where the machine-learning model is trained to identify a candidate set of resources for user selection during execution of the application builder 106 and/or during execution of a custom application. Further, as described above, operations performed during execution of the custom application may comprise presenting information associated with patient data artifacts to obtain treatment guidance.

The user-relevant information may correspond to a treatment session context, such as a role of a caregiver or caregiver specialty (e.g., cardiologist, urologist, social worker, etc.), or to a treatment venue, such as a research hospital, walk-in clinic, emergency room, or one or more conditions or clinical decision support events associated with the patient. Accordingly, in some embodiments, the user-relevant information that may be presented via execution of a particular custom application will vary, flex, or differ based on the selections made by the particular user during execution of the application builder 106 during creation by the particular user of the particular custom application and also based on the context of a particular execution of the particular custom application. For example, execution of a custom application created for a cardiologist treating a heart-attack victim in an urgent care or emergency room setting may cause presentation of a particular type of patient information such as patient vitals, medications, and previous heart condition diagnoses according to the custom application created for the cardiologist and according to the cardiologist's execution of the custom application. On the other hand, execution of another custom application created for an endocrinologist treating a diabetic patient at a research hospital may cause presentation of data according to the other custom application associated with particular needs of the endocrinologist and according to the endocrinologist's particular execution of the other custom application. For instance, execution of the other custom application may cause presentation of data, e.g., from one or both of the EHR system 108 and the EHR system 110, associated with or in addition to (augmented with) clinical studies in which the patient is enrolled and patient compliance with prescribed condition management regimens.

In some embodiments of the disclosure, software routines or software agents, e.g., electronic models, electronic adaptive agents, and/or electronic models including electronic adaptive agents, facilitate part or all of any one or more of the Operations 202-214, such as automatically adding information or user options for any of the user interfaces 310-330. In an example embodiment, the software agent(s) or software routine(s) may auto-populate or auto-configure a displayed menu a displayed field or add a text box (or drop down menu) for additional information entry (or selection) based on, e.g., prior use of the application builder 106 and/or based on an identity associated with a user or a user device. In some embodiments of the disclosure, an “auto complete” field may be presented in association with one or more of the user interfaces 310-330, e.g., providing feedback to the user indicating either that all of the information of a particular user interface has been provided by the user (YES) or that there are fields in which information is missing or incorrectly presented by the user (such as a number or letter other than V/N entered into a field requiring only a “Y” or “N”).

### 4. EXAMPLE EMBODIMENT

A detailed example is described below for purposes of clarity. Components and/or operations described below should be understood as one specific example which may not be applicable to certain embodiments of the disclosure. Accordingly, components and/or operations described below should not be construed as limiting the scope of any of the claims.

Presently, if a clinician using an EHR wishes to acquire a SMART-based software application for a specific workflow associated with the clinician, there often exists no option for the user except to make a request to the EHR system provider and hope it will be built or be built in a timely fashion. Processes involved to materialize this request can be extremely time-consuming, require a variety of resources, pose integration difficulties, and possibly add substantial cost to the user's contemplated workflow. The present disclosure allows a non-developer (clinician) to define, via a custom application, clinical information representations in a variety of ways, tables, summarizations, and sub-summarizations according to individual, e.g., unique, needs or objectives of the user.

This disclosure allows users to create and store, e.g., at the data repository 120, a library of reusable resources, components, and GUI controls (such as Drag and Drop GUI builders). For example, one or more of the computing system, the user device 102, the EHR system 108, or another component (e.g., associated with one or both of the application builder 106 or the data repository 120) may generate code for a custom application (e.g., substantially, entirely, or partially) by developing functions, routines, or particular API calls based on functions, routines, or API calls stored in the data repository 120. In some embodiments, one or more of the computing system, the user device 102, the EHR system 108, or the other component may partially or entirely generate code for a custom application by retrieving functions, routines, or particular API calls (and/or by referencing functions, routines, or particular API calls) stored in the data repository 120. During development of the custom application, embodiments of the application builder 106 automatically generate code corresponding to needed FHIR calls, e.g., based on the HL7 FHIR standard, to enable fetching of data during execution of the custom application. Based on selections made by the user, the code generator 130 of the disclosure generates needed API calls, e.g., FHIR calls, to retrieve and process user-selected data to be displayed during subsequent execution of a custom application. Hence, embodiments of the present disclosure harness mappings to FHIR concepts as indicated or needed.

Turning briefly to FIGS. 3A-3C, example user interfaces are shown for receiving information, from a user, concerning the operation, access, and parameter settings to be used by a custom application to be developed. In particular, the figures depict example user interfaces for receiving user inputs and selections, e.g., via a user device 102, specifying preferences and particulars, which are to be included in the functions and capabilities of a custom application under development by the application builder 106. Information input to the application builder via the user interfaces (e.g., that may include one or more of the first, second, additional, further, and third menus, or any portions thereof) enable a user to create a custom application with little to no code writing on the part of the user, the custom application once created being configured to access, process, and present data associated with one or more particular health care provider facilities or particular EHR systems.

FIG. 3A shows an example user interface 310 that may be presented, following selection (e.g., via the user device 102 being operated by a user) of a particular health care provider facility or EHR system, including displayed options available to a user for selecting resources and resource parameters for the custom application to be created. Based on a selection received, e.g., at Operation 202, of a particular health care provider facility or EHR system and based on analyzing metadata, e.g., at Operation 204, associated with the particular health care provider facility or EHR system to determine resources that may be available to the custom application once created, the application builder 106 may determine that resources including a “User” resource and a “Patient” resource corresponding to that shown at the top of the user interface 310 may be presented. The user interface 310 may be considered as including or corresponding to a combination of a second menu (cf. the operations of FIG. 2 just prior to Operation 206) and a third menu (cf. Operation 210) presented in a single display or alternatively presented in two consecutive displays, e.g., in response to and based on metadata determined by the application builder 106 to be associated with a selected health care provider facility or EHR system.

In the illustrated embodiment of the disclosure, separate selection options, e.g., checkboxes, are not shown for the “User” and “Patient” resources. Embodiments of the disclosure may present the user interface 310 as an additional menu (discussed above) following presentation of a second menu (discussed above) that resulted in user selections of the “User” and “Patient” resources. In other embodiments of the disclosure, a default setting of the application builder 106 causes presentation of the “User” and “Patient” resources, e.g., as automatically selected resources, in an initial user interface, e.g., corresponding to a second menu (cf. the operations of FIG. 2 just prior to Operation 206) and a third menu (as in Operation 210) based on the two resources being determined to be available for the selected health care provider facility or EHR system.

FIG. 3B shows another example user interface 320 that may be presented following selection (e.g., via the user device 102 being operated by a user) of one or more of a particular health care provider facility or an EHR system. The user interface 320 presents user options available for selection (e.g., via the user device 102 being operated by a user) of elements including “Resource Type” and “Resource Parameter.” Selection of a resource type from the Resource Type drop-down menu of resource types from which to choose (e.g., based on the application builder 106 analyzing metadata as discussed herein) causes population, by the application builder 106 based at least on metadata associated with the selected resource type, of the Resource Parameter drop-down menu with particular resource parameters relevant to the selected resource type. Further, the “Select Operator” buttons and the “Formula” field(s) in the user interface 320 allow a user to associate selected resource types and resource parameters (e.g., collectively “Operands”) into formulas (e.g., based on selected “Operators”) for processing and presenting, e.g., displaying, during execution of the custom application based on data available (in the selected health care provider facility or EHR system) corresponding to selected resources and associated information including parameter values.

As shown in FIG. 3B, a user has entitled the current formula, e.g., calculator, being constructed as “Fibrosis-4” based on the entering, e.g., typing of the letters of the title into the “Naming” or “Name of Calculator” field located at the top of the user interface 320. The user interface 320 is configured to facilitate definition by the user of one or more user-customized calculators, e.g., the “Fibrosis-4” calculator, by defining a particular formula, e.g., association of variables to be implemented by the formula, for each user-customized calculator. The example calculator displayed is configured to enable processing, by a subsequently created custom application, of information based on data extracted during execution of the custom application from one or more selected, e.g., at Operation 202, health care provider facilities or EHR systems, e.g., one or both of EHR system 108 and EHR system 110. In the example embodiment, the formula for the in-development calculator, which as shown is in the process of being defined, is shown in the “Fibrosis-4 Calculator Formula” window as “(‘Age*AST)/(’”; the formula will be enabled for use during execution of the custom application based on the code generated by the application builder 106.

FIG. 3C depicts an example user interface 330 configured to allow a user to select display options or configuration options that will be used by a subsequently created custom application during execution for presenting data retrieved from or otherwise associated with the selected health care provider facility or EHR system, e.g., data extracted from one or both of EHR system 108 and EHR system 110. In the example illustration, a user has selected, for “Number of Pages,” one page; the user further has selected, for “Number of Rows” and “Number of Columns,” two rows and two columns, respectively. As shown in the example configuration of the use interface 330, the user has defined field (0,0) to present, by the custom application during execution, data from the selected health care provider facility or EHR system extracted and processed according to the “Fibrosis-4” calculator, e.g., as defined via the user interface 320.

According to the illustrated embodiment, additional calculators such as “Observation|VITAL SIGN (BMI),” “MedicationRequest,” and “Observation|LAB (Creatinine)” are defined, e.g., based on a user interacting with the user interface 320 and user interface 330, to be presented by the custom application during execution. The additional calculators, “Observation|VITAL SIGN (BMI),” “MedicationRequest,” and “Observation|LAB (Creatinine),” will be presented in fields (0,1), (1,0), and (1,1), respectively, by the custom application during execution. In addition or alternatively, other calculators (e.g., routines), objects, or other processes defined (e.g., via the user device 102 being operated by a user) or obtained elsewhere may be selected or incorporated by a user creating the custom application for use during execution of the custom application. As an example, the other routines, objects, or processes may correspond to, e.g., include, any application available, e.g., or deemed useful for the user's intended purposes in creating the custom application, to the user or capable of being made available by the user for use by the custom application. Such routines may include items or objects as described herein such as the algorithms, applications, BDI software models, components, components embodied as part of or within other components, computer-performed services, elements, functional entities, functions, ML algorithms, modules, operations, processes, processes corresponding to hardware, firmware, and/or software, programs, routines, agents, and supervised or unsupervised components.

### 5. COMPUTER NETWORKS AND CLOUD NETWORKS

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process, e.g., initiated at user device 102, makes a request for a computing service (such as, execution of a particular application, e.g., the application builder 106, a custom application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or various applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as, a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, a particular application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment of the disclosure, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an API.

In an embodiment of the disclosure, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis. Network resources assigned to each request and/or client may be scaled up or down based on, for example, (a) the computing services requested by a particular client, (b) the aggregated computing services requested by a particular tenant, and/or (c) the aggregated computing services requested of the computer network. Such a computer network may be referred to as a “cloud network.”

In an embodiment of the disclosure, a service provider provides a cloud network to one or more end users. Various service models may be implemented by the cloud network, including but not limited to Software-as-a-Service (Saas), Platform-as-a-Service (PaaS), and Infrastructure-as-a-Service (IaaS). In SaaS, a service provider provides end users the capability to use the service provider's applications, which are executing on the network resources. In PaaS, the service provider provides end users the capability to deploy their own applications onto the network resources. The user-deployed applications may be created using programming languages, libraries, services, and tools supported by the service provider. In IaaS, the service provider provides end users the capability to provision processing, storage, networks, and other fundamental computing resources provided by the network resources. Any arbitrary applications, including an operating system, may be deployed on the network resources.

In an embodiment of the disclosure, various deployment models may be implemented by a computer network, including but not limited to a private cloud, a public cloud, and a hybrid cloud. In a private cloud, network resources are provisioned for exclusive use by a particular group of one or more entities (the term “entity” as used herein refers to a corporation, organization, person, or other entity). The network resources may be local to and/or remote from the premises of the particular group of entities. In a public cloud, cloud resources are provisioned for multiple entities that are independent from each other (also referred to as “tenants” or “customers”). The computer network and the network resources thereof are accessed by clients corresponding to different tenants. Such a computer network may be referred to as a “multi-tenant computer network.” Several tenants may use a same particular network resource at different times and/or at the same time. The network resources may be local to and/or remote from the premises of the tenants. In a hybrid cloud, a computer network comprises a private cloud and a public cloud. An interface between the private cloud and the public cloud allows for data and application portability. Data stored at the private cloud and data stored at the public cloud may be exchanged through the interface. Applications implemented at the private cloud and applications implemented at the public cloud may have dependencies on each other. A call from an application at the private cloud to an application at the public cloud (and vice versa) may be executed through the interface.

In an embodiment of the disclosure, tenants of a multi-tenant computer network are independent of each other. For example, a business or operation of one tenant may be separate from a business or operation of another tenant. Different tenants may demand different network requirements for the computer network. Examples of network requirements include processing speed, amount of data storage, security requirements, performance requirements, throughput requirements, latency requirements, resiliency requirements, Quality of Service (QoS) requirements, tenant isolation, and/or consistency. The same computer network may need to implement different network requirements demanded by different tenants.

In one or more embodiments, in a multi-tenant computer network, tenant isolation is implemented to ensure that the applications and/or data of different tenants are not shared with each other. Various tenant isolation approaches may be used.

In an embodiment of the disclosure, each tenant is associated with a tenant ID. Each network resource of the multi-tenant computer network is tagged with a tenant ID. A tenant is permitted access to a particular network resource only if the tenant and the particular network resources are associated with a same tenant ID.

In an embodiment of the disclosure, each tenant is associated with a tenant ID. Each application, implemented by the computer network, is tagged with a tenant ID. Additionally or alternatively, each data structure and/or dataset, stored by the computer network, is tagged with a tenant ID. A tenant is permitted access to a particular application, data structure, and/or dataset only if the tenant and the particular application, data structure, and/or dataset are associated with a same tenant ID.

As an example, each database implemented by a multi-tenant computer network may be tagged with a tenant ID. Only a tenant associated with the corresponding tenant ID may access data of a particular database. As another example, each entry in a database implemented by a multi-tenant computer network may be tagged with a tenant ID. Only a tenant associated with the corresponding tenant ID may access data of a particular entry. However, the database may be shared by multiple tenants.

In an embodiment of the disclosure, a subscription list indicates which tenants have authorization to access which applications. For each application, a list of tenant IDs of tenants authorized to access the application is stored. A tenant is permitted access to a particular application only if the tenant ID of the tenant is included in the subscription list corresponding to the particular application.

In an embodiment of the disclosure, network resources (such as digital devices, virtual machines, application instances, and threads) corresponding to different tenants are isolated to tenant-specific overlay networks maintained by the multi-tenant computer network. As an example, packets from any source device in a tenant overlay network may only be transmitted to other devices within the same tenant overlay network. Encapsulation tunnels are used to prohibit any transmissions from a source device on a tenant overlay network to devices in other tenant overlay networks. Specifically, the packets, received from the source device, are encapsulated within an outer packet. The outer packet is transmitted from an encapsulation tunnel endpoint (in communication with the source device in the tenant overlay network) to another encapsulation tunnel endpoint (in communication with the destination device in the tenant overlay network). The other encapsulation tunnel endpoint decapsulates the outer packet to obtain the original packet transmitted by the source device. The original packet is transmitted from the other encapsulation tunnel endpoint to the destination device in the same particular overlay network.

### 6. MICROSERVICE APPLICATIONS

According to one or more embodiments, the techniques described herein are implemented in a microservice architecture. A microservice in this context refers to software logic designed to be independently deployable, having endpoints that may be logically coupled to other microservices to build a variety of applications. Applications built using microservices are distinct from monolithic applications, which are designed as a single fixed unit and generally comprise a single logical executable. With microservice applications, different microservices are independently deployable as separate executables. Microservices may communicate using HyperText Transfer Protocol (HTTP) messages and/or according to other communication protocols via API endpoints. Microservices may be managed and updated separately, written in different languages, and be executed independently from other microservices.

Microservices provide flexibility in managing and building applications. Different applications may be built by connecting different sets of microservices without changing the source code of the microservices. Thus, the microservices act as logical building blocks that may be arranged in a variety of ways to build different applications. Microservices may provide monitoring services that notify a microservices manager (such as If-This-Then-That (IFTTT), Zapier, or Oracle Self-Service Automation (OSSA)) when trigger events from a set of trigger events exposed to the microservices manager occur. Microservices exposed for an application may alternatively or additionally provide action services that perform an action in the application (controllable and configurable via the microservices manager by passing in values, connecting the actions to other triggers and/or data passed along from other actions in the microservices manager) based on data received from the microservices manager. The microservice triggers and/or actions may be chained together to form recipes of actions that occur in optionally different applications that are otherwise unaware of or have no control or dependency on each other. These managed applications may be authenticated or plugged in to the microservices manager, for example, with user-supplied application credentials to the manager, without requiring reauthentication each time the managed application is used alone or in combination with other applications.

### Triggers

The techniques described above may be encapsulated into a microservice, according to one or more embodiments. In other words, a microservice may trigger a notification (into the microservices manager for optional use by other plugged in applications, herein referred to as the “target” microservice) based on the above techniques and/or may be represented as a GUI block and connected to one or more other microservices. A user may connect the output of one microservice into the input of another microservice using directed arrows or any other GUI element, whereby for example the computing system or the application builder 106 may run verification tests to confirm that the output and inputs are compatible, e.g., by checking the data types or size restrictions.

The trigger condition may include absolute or relative thresholds for values, and/or absolute or relative thresholds for the amount or duration of data to analyze, such that the trigger to the microservices manager occurs whenever a plugged-in microservice application detects that a threshold is crossed. For example, a user may request a trigger into the microservices manager when the microservice application detects a value has crossed a triggering threshold.

In one embodiment of the disclosure, the trigger, when satisfied, might output data for consumption by the target microservice. In another embodiment of the disclosure, the trigger, when satisfied, outputs a binary value indicating the trigger has been satisfied, or outputs the name of the field or other context information for which the trigger condition was satisfied. Additionally or alternatively, the target microservice may be connected to one or more other microservices such that an alert is input to the other microservices. Other microservices may perform responsive actions based on the above techniques, including, but not limited to, deploying additional resources, adjusting system configurations, and/or generating GUIs.

### Actions

In one or more embodiments, a plugged-in microservice application may expose actions to the microservices manager. The exposed actions may receive, as input, data or an identification of a data object or location of data, that causes data to be moved into a data cloud.

In one or more embodiments, the exposed actions may receive, as input, a request to increase or decrease existing alert thresholds. The input might identify existing in-application alert thresholds and whether to increase or decrease, or delete the threshold. Additionally or alternatively, the input might request the microservice application to create new in-application alert thresholds. The in-application alerts may trigger alerts to the user while logged into the application, or may trigger alerts to the user using default or user-selected alert mechanisms available within the microservice application itself, rather than through other applications plugged into the microservices manager.

In one or more embodiments, the microservice application may generate and provide an output based on input that identifies, locates, or provides historical data, and defines the extent or scope of the requested output. The action, when triggered, causes the microservice application to provide, store, or display the output, for example, as a data model or as aggregate data that describes a data model.

### 7. HARDWARE OVERVIEW

According to one embodiment of the disclosure, the techniques described herein are implemented by one or more special-purpose computing devices. The special-purpose computing devices may be hard-wired to perform the techniques, or may include: (i) digital electronic devices such as one or more application-specific integrated circuits (ASICs), field programmable gate arrays (FPGAs), or network processing units (NPUs) that are persistently programmed to perform the techniques, or (ii) one or more general purpose hardware processors programmed to perform the techniques pursuant to program instructions in firmware, memory, other storage, or a combination. Such special-purpose computing devices may also combine custom hard-wired logic, ASICS, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

For example, FIG. 4 is a block diagram illustrating the computer system 400 upon which an embodiment of the disclosure may be implemented. Computer system 400 includes a bus 402 or other communication mechanism for communicating information, and a hardware processor 404 coupled with bus 402 for processing information. Hardware processor 404 may be, for example, a general purpose microprocessor.

Computer system 400 also includes a main memory 406, such as a random access memory (RAM) or other dynamic storage device, coupled to bus 402 for storing information and instructions to be executed by processor 404. Main memory 406 also may be used for storing temporary variables or other intermediate information during execution of instructions to be executed by processor 404. Such instructions, when stored in non-transitory storage media accessible to processor 404, render computer system 400 into a special-purpose machine that is customized to perform the operations specified in the instructions.

Computer system 400 further includes a read only memory (ROM) 408 or other static storage device coupled to bus 402 for storing static information and instructions for processor 404. A storage device 410, such as a magnetic disk or optical disk, is provided and coupled to bus 402 for storing information and instructions.

Computer system 400 may be coupled via bus 402 to a display 412, such as a cathode ray tube (CRT), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, an axis (e.g., x) and another axis (e.g., y), that allows the device to specify positions in a plane.

Computer system 400 may implement the techniques described herein using customized hard-wired logic, one or more ASICs or FPGAs, firmware and/or program logic which in combination with the computer system causes or programs computer system 400 to be a special-purpose machine. According to one embodiment of the disclosure, the techniques herein are performed by computer system 400 in response to processor 404 executing one or more sequences of one or more instructions contained in main memory 406. Such instructions may be read into main memory 406 from another storage medium, such as storage device 410. Execution of the sequences of instructions contained in main memory 406 causes processor 404 to perform the process operations described herein. In alternative embodiments of the disclosure, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 410. Volatile media includes dynamic memory, such as main memory 406. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

Computer system 400 also includes a communication interface 418 coupled to bus 402. Communication interface 418 provides a two-way data communication coupling to a network link 420 that is connected to a local network 422. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN. Wireless links may also be implemented. In any such implementation, communication interface 418 sends and receives electrical, electromagnetic or optical signals that carry digital data streams representing various types of information.

Network link 420 typically provides data communication through one or more networks to other data devices. For example, network link 420 may provide a connection through local network 422 to a host computer 424 or to data equipment operated by an Internet Service Provider (ISP) 426. ISP 426 in turn provides data communication services through the world wide packet data communication network now commonly referred to as the “Internet” 428. Local network 422 and Internet 428 both use electrical, electromagnetic or optical signals that carry digital data streams. The signals through the various networks and the signals on network link 420 and through communication interface 418, which carry the digital data to and from computer system 400, are example forms of transmission media.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution.

### 8. MISCELLANEOUS; EXTENSIONS

Unless otherwise defined, all terms (including technical and scientific terms) are to be given their ordinary and customary meaning to a person of ordinary skill in the art, and are not to be limited to a special or customized meaning unless expressly so defined herein.

This application includes references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment of the disclosure, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments of the disclosure. In the foregoing specification, embodiments of the disclosure have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. A non-transitory computer readable medium comprising instructions which, when executed by one or more hardware processors, cause performance of operations comprising:
receiving a selection of a first resource defined by a first Electronic Health Record (EHR) system;
analyzing metadata associated with the first resource to identify a first set of characteristics corresponding to the first resource;
identifying one or more configuration fields associated with the first resource based on the first set of characteristics;
presenting the one or more configuration fields associated with the first resource;
receiving one or more configuration values for the one or more configuration fields; and
generating code for (a) executing Application Programming Interface (API) calls to extract data associated with the first resource in accordance with the one or more configuration values and (b) presenting the data associated with the first resource.

2. The computer readable medium of claim 1, wherein:
prior to receiving the selection, the first EHR system is analyzed to identify a plurality of resources associated with the first EHR system; and
the plurality of resources are associated with an FHIR standard, include the first resource, and are presented as a candidate set of resources for user selection.

3. The computer readable medium of claim 1, wherein the operations further comprise executing the code to (a) execute the API calls to extract the data associated with the first resource and associated with a FHIR protocol, and (b) present the data associated with the first resource in accordance with the one or more configuration values.

4. The computer readable medium of claim 3, wherein executing the code causes retrieving from the EHR system resources that correspond to patient data artifacts associated with at least one record of the patient in the EHR system.

5. The computer readable medium of claim 4, wherein the operations further comprise presenting information associated with patient data artifacts to obtain treatment guidance.

6. The computer readable medium of claim 5, wherein presenting the information includes applying an AI model to the information.

7. The computer readable medium of claim 1, wherein the EHR system is FHIR compliant and configured to be mapped to one or more of: patient medical records associated with a particular hospital, or patient medical records associated with an HIE platform and related to immunization status.

8. The computer readable medium of claim 1, the operations further comprising executing a machine-learning model, the machine-learning model trained to identify a candidate set of resources for user selection.

9. The computer readable medium of claim 1, wherein:
the one or more configuration fields are associated with user scopes, the user scopes including a plurality of resources associated with a corresponding plurality of selectable scopes; and
at least one of the plurality of selectable scopes is associated with a selectable read-access permission field and a selectable write-access permission field.

10. The computer readable medium of claim 1, wherein the one or more configuration fields are associated with a plurality of selectable user scope fields.

11. The computer readable medium of claim 1, wherein the one or more configuration fields are associated with a plurality of patient scope fields, wherein each patient scope field is associated with a resource of the EHR that is configurable via user selection to one or more of read-access enabled, read-access disabled, white-access enabled, or write-access disabled.

12. The computer readable medium of claim 1, wherein:
the selection of the first resource indicates a resource type;
in response to the selection of the first resource, the metadata associated with the first resource is analyzed to identify the first set of characteristics and to identify the one or more configuration fields; and
presenting the one or more configuration fields includes presenting: a plurality of selectable resource parameter fields, a user-definable number of instances data field, a type of display format field, and a display location of data field.

13. The computer readable medium of claim 12, wherein: the selection of the first resource indicates an Observation resource type, and the plurality of selectable resource parameter fields is included in a drop-down menu that includes the following selectable fields: VITAL SIGN (BMI), VITAL SIGN (Pressure), LAB (Creatinine), and LAB (Hemoglobin).

14. The computer readable medium of claim 1, wherein:
the selection of the first resource is based on a drop-down menu that includes a Patient resource type, an Observation resource type, a Procedure resource type, and a MedicationRequest resource type; and
in response to a selection of the Observation resource type, a plurality of selectable Resource Parameter fields associated with the Observation resource type are presented.

15. A method comprising:
receiving a selection of a first resource defined by a first Electronic Health Record (EHR) system;
analyzing metadata associated with the first resource to identify a first set of characteristics corresponding to the first resource;
identifying one or more configuration fields associated with the first resource based on the first set of characteristics;
presenting the one or more configuration fields associated with the first resource;
receiving one or more configuration values for the one or more configuration fields; and
generating code for (a) executing Application Programming Interface (API) calls to extract data associated with the first resource and (b) presenting the data associated with the first resource in accordance with the one or more configuration values.

16. The method of claim 15, wherein:
prior to receiving the selection, the first EHR system is analyzed to identify a plurality of resources associated with the first EHR system; and
the plurality of resources are associated with an FHIR standard, include the first resource, and are presented as a candidate set of resources for user selection.

17. The method of claim 15, further comprising executing the code to (a) execute the API calls to extract the data associated with the first resource and associated with an FHIR protocol, and (b) present the data associated with the first resource in accordance with the one or more configuration values.

18. A system comprising a processor and a computer-readable data storage device storing program instructions that, when executed by the processor, control the system to perform operations comprising:
receiving a selection of a first resource defined by a first Electronic Health Record (EHR) system;
analyzing metadata associated with the first resource to identify a first set of characteristics corresponding to the first resource;
identifying one or more configuration fields associated with the first resource based on the first set of characteristics;
presenting the one or more configuration fields associated with the first resource;
receiving one or more configuration values for the one or more configuration fields; and
generating code for (a) executing Application Programming Interface (API) calls to extract data associated with the first resource and (b) presenting the data associated with the first resource in accordance with the one or more configuration values.

19. The system of claim 18, wherein:
prior to receiving the selection, the first EHR system is analyzed to identify a plurality of resources associated with the first EHR system; and
the plurality of resources are associated with an FHIR standard, include the first resource, and are presented as a candidate set of resources for user selection.

20. The system of claim 18, wherein the operations further comprise executing the code to (a) execute the API calls to extract the data associated with the first resource and associated with a FHIR protocol, and (b) present the data associated with the first resource in accordance with the one or more configuration values.
